(19) United States
(12) Patent Application Publication
(10) Pub. No.: US 2011/0082171 A1
(43) Pub. Date: Apr. 7, 2011

(54) Medicaments and Methods for Inhibition of Scarring

(75) Inventors: Mark William James Ferguson, Manchester (GB); Nicholas Occleston, Manchester (GB); Sharon O'Kane, Manchester (GB); Kerry Nield, Manchester (GB); Nick Goldspink, Manchester (GB)

(73) Assignee: Renovo Limited, Manchester (GB)

(21) Appl. No.: 12/995,900

(22) PCT Filed: Jun. 2, 2009
(86) PCT No.: PCT/GB09/01386

§ 371 Date: Dec. 2, 2010

(30) Foreign Application Priority Data
Jun. 3, 2008 (GB) 08100063.8

(51) Int. Cl. A61K 31/437; A61P 17/02

(52) U.S. Cl. 514/302

(57) ABSTRACT

Provided is the use of an agonist of a GABA_{A} receptor for use in the prevention, reduction or inhibition of scarring formed on healing of a wound. Also provided is a method of preventing, reducing or inhibiting scarring formed on healing of a wound, in which a therapeutically effective amount of an agonist of a GABA_{A} receptor is administered to a patient in need of such prevention, reduction or inhibition. The GABA_{A} receptor agonist used may be an agonist specific to the GABA_{A} receptor, such as Gaboxadol (7-tetra hydroisoxazolo[5,4-c]pyridin-3-ol), or a pharmaceutically acceptable salt thereof. The scarring to be prevented, reduced or inhibited may be scarring formed on healing of a wound of the dermis.

The present invention relates to the manufacture of medicaments for the prevention, reduction or inhibition of scarring formed on healing of a wound. The invention also provides methods for the prevention, reduction or inhibition of scarring formed on healing of a wound. The invention further provides an agonist of a GABA_{A} receptor for use in the prevention, reduction or inhibition of scarring formed on healing of a wound.

Clinical approaches to wound management will generally depend on the outcome that it is desired to achieve. This outcome may, for example, be considered with reference to the degree of scarring occurring, or with reference to the speed at which a wound heals. In management of some wounds control of the degree of scarring that occurs is of primary importance, while increasing the speed of wound healing is of much lesser importance. In management of other wounds increasing the speed of wound healing is of primary importance, while controlling the degree of scarring occurring is of much lesser importance. The present invention is applicable to the management of wounds in which the primary clinical concern relates to the degree of scarring arising as a result of healing.

Many different processes are at work during the scarring response, and much research has been conducted into discovering what mediates these processes, and how they interact with each other to produce the final outcome.

The scarring response is a common result of the healing of a wound shared by all adult mammals. The scarring response is conserved between the majority of tissue types and in each case leads to the same result, formation of a reparative tissue termed a “scar”. A scar may be defined as “fibrous connective tissue that forms at the site of injury in any tissue of the body”.

A scar constitutes the structure produced as a result of the reparative response triggered by tissue injury. This reparative process has arisen as the evolutionary solution to the biological imperative to prevent the death of a wounded animal. In order to overcome the risk of mortality due to infection or blood loss, the body reacts rapidly to repair the damaged area, rather than attempt to regenerate the damaged tissue. Since the damaged tissue is not regenerated to attain the same tissue architecture present before wounding, a scar may be identified by virtue of its abnormal morphology as compared to unwounded tissue.

Although pronounced scars may have a “bulky” appearance, they are not generally associated with excessive cell proliferation in the damaged area, and often contain fewer cells than surrounding unwounded tissues. Scars are primarily composed of connective tissue, the connective tissue in question being determined by the body site at which scarring takes place. This material is deposited by cells involved in the healing process, which may be derived from the injured tissue, from tissues surrounding the injured tissue, or from the circulation. A scar may comprise connective tissue that has an abnormal organisation, as is frequently observed in scars of the skin. Alternatively or additionally, a scar may comprise connective tissue that is present in an abnormally increased amount. Most scars consist of both abnormally organised and excess connective tissue, as described further below.

The abnormal structure of scars may be observed with reference to both their internal structure (which may be determined by means of microscopic analysis) and their external appearance (which may be assessed macroscopically).

In connective tissues, such as the skin, extracellular matrix (ECM) molecules (e.g. collagen, fibronectin etc) comprise the major structural component of both “normal” (unwounded) and scarred tissues. In normal skin these molecules form fibres which, when viewed microscopically, have a characteristic random arrangement that is commonly referred to as “basket-weave”. This basket-weave arrangement is disrupted in scars. ECM fibres in scars exhibit a marked degree of alignment with each other as compared to the random arrangement of fibres in normal skin. In general the fibres observed within scars are also of smaller diameter than those seen in normal skin. Both the size and arrangement of ECM fibres may contribute to the scars altered mechanical properties, most notably increased stiffness, when compared with normal skin.

Viewed macroscopically, scars may be depressed below the surface of the surrounding tissue, or elevated above the surface of their undamaged surroundings. Scars may be relatively darker coloured than normal tissue (hyperpigmentation) or may have a paler colour (hypopigmentation) compared to their surroundings. In the case of scars of the skin, either hyperpigmented or hypopigmented scars constitute a readily apparent cosmetic defect. It is also known that scars of the skin may be redder than unwounded skin, causing them to be noticeable and cosmetically unacceptable. It has been shown that the cosmetic appearance of a scar is one of the major factors contributing to the psychological impact of scars upon the sufferer, and that these effects can remain long after the injury itself has passed.

In addition to their psychological effects, scars may also have deleterious physical effects upon the sufferer. These effects typically arise as a result of the mechanical differences between scars and normal tissue. The abnormal structure and composition of scars mean that they are typically less flexible than their normal tissue counterpart. As a result scars may be responsible for impairment of normal function (such as in the case of scars covering joints which may restrict the possible range of movement) and may retard normal growth if present from an early age.

Scars occur at many body sites, and the effects of scarring at these sites will generally be related to loss or disruption of function in the scarred area. Some of the disadvantages associated with scarring of the skin have been discussed above. Scarring in the eye (for example, as a result of accidental injury or surgical intervention) can impair vision and even lead to blindness. Scarring of the internal organs may lead to the formation of strictures and adhesions that significantly or totally impair function of the organ in question. Scarring of tendons and ligaments may cause lasting damage to these organs, and thereby reduce the motility or function of associated joints. Scarring associated with blood vessels, and particularly the valves of the heart, may occur after injury or surgery. Scarring of blood vessels may lead to restenosis, which causes a narrowing of the blood vessel and thus reduces the flow of blood through the scarred area.

The effects outlined above may all arise as a result of the normal progression of the wound healing response. There are, however, many ways in which the scarring response may be abnormally altered; and these are frequently associated with even more damaging effects resulting from the production of abnormal excessive scarring (commonly referred to as pathological scarring). There are a number of methods by which pathological scarring may be differentiated from severe scarring resulting from the normal healing response. These include histological differences in the scars produced, as well as genetic markers that may indicate a disposition to pathological scarring. An individual's history of pathological or non-pathological scarring remains one of the most effective predictors of the likelihood of future incidences of pathological scarring. The most frequent and important classes of pathological scarring include hypertrophic scarring, keloid scarring and pterygium.

Whilst much of the present specification concentrates primarily on the effects of scarring in man, it will be appreciated that many aspects of the scarring response are conserved between most species of animals. Thus, the problems outlined above are also applicable to non-human animals, and particularly veterinary or domestic animals (e.g. horses, cattle, dogs, cats etc). By way of example, it is well known that adhesions resulting from the inappropriate healing of abdominal wounds constitute a major reason for the veterinary destruction of horses (particularly race horses). Similarly the tendons and ligaments of domestic or veterinary animals are also frequently subject to injury, and healing of such injuries may also lead to scarring associated with increased animal mortality.

Although the ill effects of scarring resulting from the healing of wounds are well known there remains a lack of effective therapies able to reduce these effects. In the light of this absence it must be recognised that there exists a strong need to provide medicaments and treatments that are able to prevent, reduce or inhibit scarring formed on healing of a wound.

GABA receptors are a class of cellular receptors that respond to the neurotransmitter gamma-aminobutyric acid (GABA). GABA is the major inhibitory neurotransmitter in the central nervous systems of vertebrates.

GABA receptors may be assigned to one of three classes: GABA_{A} receptors, GABA_{B} receptors or GABA_{C} receptors. GABA_{A} and GABA_{C} receptors are both ligand-gated Cl⁻ ion channel receptors, while GABA_{B} receptors are G protein-coupled receptors. GABA_{A} receptors are multimeric transmembrane receptors with five subunits arranged around a central pore. There are numerous subunit isoforms for the GABA_{A} receptor. In humans six types of α subunit are known, with three forms of the β subunit, three forms of the γ subunit, as well as a δ, an ε, a π and a θ subunit.

It is an aim of certain aspects of the present invention to provide medicaments suitable for the prevention and/or reduction and/or inhibition of scarring formed on healing of a wound. It is an aim of further aspects of the present invention to provide methods of treatment suitable for use in the prevention, and/or reduction, and/or inhibition of scarring formed on healing of a wound. It is an aim of certain embodiments of the invention to provide new uses of GABA_{A} receptor agonists. The medicaments and/or methods and or uses of the invention may constitute alternatives to those provided by the prior art. However, it is preferred that medicaments and/or methods and/or uses provided by the invention may constitute improvements over the prior art.

According to a first aspect of the present invention there is provided an agonist of a GABA_{A} receptor for use in the prevention, reduction or inhibition of scarring formed on healing of a wound. The use may comprise administration of the GABA_{A} receptor agonist at a site where scarring is to be prevented, reduced or inhibited. The use may comprise administration of the GABA_{A} receptor agonist at a wound, or a site where a wound is to be formed.

In a second aspect of the invention there is provided the use of an agonist of a GABA_{A} receptor in the manufacture of a medicament for use in the prevention, reduction or inhibition of scarring formed on healing of a wound. The medicament may be a topical medicament for application at a site where scarring is to be prevented, reduced or inhibited. The medicament may preferably be for use at a wound, or at a site where a wound is to be formed. Medicaments may be manufactured in accordance with this aspect of the invention based on any of the information included in this specification, and suitable for putting into practice any of the uses or methods of the invention described herein.

In a third aspect of the invention there is provided a method of preventing, reducing or inhibiting scarring formed on healing of a wound, the method comprising administering a therapeutically effective amount of an agonist of a GABA_{A} receptor, to a patient in need of such prevention, reduction or inhibition. The GABA_{A} receptor agonist, may preferably be administered to the site where scarring is to be prevented, reduced or inhibited. The site may preferably be a wound, or a site where a wound is to be formed.

GABA_{A} receptor agonists suitable for use in accordance with the invention are discussed in greater detail elsewhere in the specification. Preferred GABA_{A} receptor agonists suitable for such use may be selected from the group consisting of: Gaboxadol (7-tetra hydroisoxazolo[5,4-c]pyridin-3-ol; also known by the abbreviation THIP); muscimol; isoguvacine; isonipecotic acid; and piperidine-4-sulphonic acid. Of this group Gaboxadol represents a particularly preferred GABA_{A} receptor agonist for use in accordance with the invention. Gaboxadol may be used as the only GABA_{A} receptor agonist employed in accordance with the invention, or may be use in combination with one, or more, other GABA_{A} receptor agonists. That said, it may be preferred that the medicaments or methods of the invention utilise a GABA_{A} receptor agonist other than Gaboxadol.

It may generally be preferred that a GABA_{A} receptor for use in the accordance with the invention (whether in the first, second or third aspects) comprises a specific GABA_{A} receptor agonist. Specific GABA_{A} receptor agonists are also discussed in more detail elsewhere in the specification.

The inventors believe that the prevention, reduction or inhibition of scarring using a GABA_{A} receptor agonist may be effected at any body site, and in any tissue or organ, where a wound may occur. However, the inventors believe that the anti-scarring effect observed in the skin is particularly significant, and so skin represents a preferred organ in which scarring formed on healing of a wound may be prevented, reduced or inhibited in accordance with the invention. Accordingly, it will be recognised that skin wounds, or sites where skin wounds are to be formed, may beneficially be treated using the medicaments or methods of the invention.

Various terms that are used in the present disclosure to describe the invention will now be explained further. The definitions and guidance provided below may be expanded on elsewhere in the specification as appropriate, and as the context requires.

### “Agonist of a GABA_{A} Receptor”

For the purpose of the present disclosure an “agonist of a GABA_{A} receptor” (also referred to as a “GABA_{A} receptor agonist” for purposes of brevity) should be taken, except for where the context requires otherwise, as encompassing any agonist of the GABA_{A} class of receptors, provided that the agonist is capable of inhibiting scarring. Preferred means by which inhibition of scarring achieved by GABA_{A} receptor agonists may be assessed (and quantified if required) are considered elsewhere in the specification, and include the use of visual analogue scales relating to macroscopic and/or microscopic scarring.

The skilled person will be aware of many GABA_{A} receptor agonists (in addition to GABA itself) that may be suitable for use in accordance with the invention. The following paragraphs provide non-limiting guidance as to the selection of suitable GABA_{A} receptor agonists, and suggestions as to particular GABA_{A} receptor agonists that may be used in accordance with the invention.

Gaboxadol (also known as 4,5,6,7-tetrahydroisoxazolo[5,4-c]pyridine-3-ol, or THIP) is a preferred example of a specific GABA_{A} receptor agonist suitable for use in accordance with the present invention. Gaboxadol is derived from ibotenic acid (a naturally occurring neuroactive 3-isoxazole derived from the mushroom Amanita muscaria). When Amanita muscaria is dried, ibotenic acid is decarboxylated to produce muscimol, a conformationally restricted analog of GABA in which a hydroxyisoxazole moiety replaces the carboxyl group of GABA. Muscimol is a potent GABA_{A} receptor agonist with a 10-fold greater affinity than GABA at the GABA_{A} receptor. Gaboxadol is derived from one of the possible conformations of muscimol and shares muscimol's high affinity for the GABA_{A} receptor. Other muscimol derivatives such as thiomuscimol and dihydromuscimol; isonipecotic acid and piperidine-4-sulfonic acid (P4S) also act as GABA_{A} receptor agonists.

While muscimol derivatives constitute a useful class of GABA_{A} receptor agonists that may be used in accordance with the present invention, the practice of the invention is not limited to the use of such compounds, and many other suitable GABA_{A} receptor agonists will be known to those skilled in the art.

Merely by way of example, the inventors believe that GABA_{A} receptor agonists selected from the group consisting of: Gaboxadol; muscimol; thiomuscimol; dihydromuscimol; isonipecotic acid; piperidine-4-sulfonic acid; isoguvacine hydrochloride; 3-amino-1-propanesulfonic acid sodium salt; chlormethiazole hydrochloride; and DEABL (3,3-diethyl-2-pyrrolidinone diethyl-lactam) may show anti-scarring properties, and so be suitable for use in the present invention. Many of these GABA_{A} receptor agonists are available commercially (for example, from Sigma-Aldrich).

The state of the art also provides guidance to the skilled person as to how novel GABA_{A} receptor agonists may be designed. Such novel GABA_{A} receptor agonists may be suitable for use in accordance with the present invention. Merely by way of example, techniques that may be useful in the design of novel GABA_{A} receptor agonists to be used in accordance with the present invention may be found in the publication “Specific GABA_{A} agonists and partial agonists” by Krogsgaard-Larsen P, Frolund B, Liljefors T (Chem Rec 2002, 2:419-430). Many GABA_{A} receptor agonists are now known that have an affinity for GABA_{A} receptors that is greater than that of GABA, and it may be preferred to use such agonists in the present invention. Gaboxadol is an example of a GABA_{A} receptor agonist with an affinity higher than that of GABA itself. For example, Brown et al. (British Journal of Pharmacology 2002, 136(7):965-74) demonstrated that Gaboxadol acts with high potency (EC₅₀ of 6 μM) and demonstrates an increased maximum response (163% of that of GABA) at GABA_{A} receptors containing α4β3δ subunits.

It may generally be preferred that a specific GABA_{A} receptor agonist be used in accordance with the invention. A “specific” GABA_{A} receptor agonist may be defined as an agent that agonises GABA_{A} receptors, but does not agonise GABA_{B} or GABA_{C} receptors.

It may be preferred that the GABA_{A} receptor agonist Gaboxadol is used in the form of a pharmaceutically acceptable salt. Such salts may be more readily manufactured and handled than Gaboxadol alone. Gaboxadol hydrochloride (the hydrochloric acid salt of Gaboxadol) represents a preferred form of Gaboxadol for use in accordance with the present invention. Except for those occasions on which the context requires otherwise, references to Gaboxadol in the present specification should be taken to encompass any pharmaceutically acceptable salt in general, and the hydrochloric acid salt in particular.

In the event that the relevant art provides no indication as to whether or not a compound of interest is a GABA_{A} receptor agonist, the ability of the compound to activate the GABA_{A} receptor may be investigated using any suitable assays known to the skilled person. These include, but are not limited to, assays described in Wafford & Ebert (Current Opinion in Pharmacology 2006, 6:30-36) and Stórustovu & Ebert (The Journal of Pharmacology and Experimental Therapeutics 2006, 316:1351-1359) in which cloned GABA_{A} receptors, expressed in oocytes (particularly Xenopus oocytes), are used to evaluate the efficacy of GABA_{A} receptor agonists.

The disclosures of these documents, in particular insofar as they relate to assays, by which compounds of interest may be assessed for GABA_{A} receptor agonist activity, are incorporated by reference.

A therapeutically effective GABA_{A} receptor agonist suitable for use in the medicaments or methods of the invention may be an agonist that is effective to inhibit scarring by at least 10% compared to a suitable control. Preferably a therapeutically effective GABA_{A} receptor agonist may be capable of inhibiting scarring by at least 20%, more preferably at least 50%, even more preferably at least 75% and yet more preferably by at least 90% compared to a suitable control. A most preferred therapeutically effective GABA_{A} receptor agonist may be capable of inhibiting scarring by 100% as compared to a suitable control.

In particular, therapeutically effective GABA_{A} receptor agonists, such as Gaboxadol, suitable for use in the medicaments or methods of the invention may be those able to alter the amount and/or orientation of extracellular matrix components (such as collagen) present in a treated scar and thereby inhibit scarring. A therapeutically effective GABA_{A} receptor agonist suitable for use in the medicaments or methods of the invention may be one that is able to give rise to a treated scar in which the ECM architecture is more like that of unwounded tissue.

Preferably a therapeutically effective GABA_{A} receptor agonist, such as Gaboxadol, may be one that is capable of inhibiting scarring at a site to which the agonist is administered. Such a site may be a wound, or a site where a wound is to be formed.

Generally, it may be preferred that GABA_{A} receptor agonists to be used in the medicaments or methods of the invention are agonists having a relatively long half life in the body of a patient to whom the agonist is administered.

It will be appreciated that a mixture of two, or more, different GABA_{A} receptor agonists may be used in the medicaments or methods of the invention to inhibit scarring formed on healing of a wound. Indeed, such use may represent a preferred embodiment of the invention. Mixtures of different GABA_{A} receptor agonists suitable for use in this manner may include GABA_{A} receptor agonists capable of agonising more than one sub type of GABA_{A} receptor (i.e. with different α, β, γ, δ or ε subunit composition), or may alternatively comprise a mixture of GABA_{A} receptor agonists sharing a specificity for the same subtype class of GABA_{A} receptors.

### “Therapeutically Effective Amounts”

A therapeutically effective amount of a GABA_{A} receptor agonist, such as Gaboxadol, is any amount of such a GABA_{A} receptor agonist that is able to prevent, reduce or inhibit scarring formed on healing of a wound.

A therapeutically effective amount of a GABA_{A} receptor agonist is preferably an amount able to inhibit scarring at a site to which the GABA_{A} receptor agonist is administered. Such a site may be a site where a wound is to be formed, or may be a wound.

A therapeutically effective amount of a medicament of the invention is any amount of a medicament of the invention that is able to inhibit scarring. This inhibition of scarring may preferably be achieved at a site to which the medicament of the invention is administered.

The skilled person will appreciate that a GABA_{A} receptor agonist that has little inherent therapeutic activity will still be therapeutically effective if administered in a quantity that provides a therapeutically effective amount.

A therapeutically effective amount of a GABA_{A} receptor agonist for use in the medicaments or methods of the invention may be preferably be an amount that is able to stimulate GABA_{A} receptors at a site where the GABA_{A} receptor agonist is to have therapeutic activity.

The inventors have found that therapeutically effective amounts of active agents able to inhibit scarring in vivo in animal models (such as rat incisional wound models) are generally also effective to inhibit scarring in human subjects. This is particularly the case for active agents administered locally. The inventors have found that therapeutically effective amounts identified in animal models are frequently directly comparable to therapeutically effective amounts that may be used in humans. Although they do not wish to be bound by any hypothesis, the inventors believe that this may be due to the decreased influence of factors such as rates of metabolic clearance of active agents by human or non-human animals when localised administration (particularly to organs such as the skin) is compared with systemic administration (in which clearance of the active agents from the circulation by the liver can play a major role in determining biological effectiveness of such agents).

A therapeutically effective amount of a GABA_{A} receptor agonist, such as Gaboxadol, or of a medicament of the invention, may preferably be an amount of an agonist or medicament that is effective to inhibit scarring by at least 10% compared to a relevant control. Preferably a therapeutically effective amount of a GABA_{A} receptor agonist, or of a medicament of the invention, may be capable of inhibiting scarring by at least 20%, more preferably at least 50%, even more preferably at least 75% and yet more preferably of inhibiting scarring by at least 90% compared to a relevant control. A most preferred therapeutically effective amount of a GABA_{A} receptor agonist, or a medicament of the invention, may be capable of inhibiting scarring by 100% as compared to a relevant control.

The selection of a suitable control will be apparent to one skilled in the art, but by way of guidance, in the event that it is wished to assess inhibition of scarring on healing of wounds treated with a GABA_{A} receptor agonist, a suitable control may comprise an untreated or control treated wound.

Thus, a therapeutically effective amount of a GABA_{A} receptor agonist, such as Gaboxadol, or of a medicament of the invention, may be an amount that is effective to reduce scarring occurring on healing of a treated wound by at least 10% compared to scarring occurring on healing of an untreated or control wound. “Treated wounds” and “untreated wounds” or “control wounds” are defined elsewhere in the specification. Preferably a therapeutically effective amount of a GABA_{A} receptor agonist, or of a medicament of the invention, may be capable of causing a 20% inhibition of scarring, more preferably at least a 50% inhibition, even more preferably at least a 75% inhibition and most preferably at least a 90% inhibition of the scarring occurring on healing of a treated wound as compared to scarring occurring on healing of an untreated or control wound.

Suitable experimental models of scarring associated with healing of wounds, and suitable controls that may be used when assessing scarring occurring in such models, are considered elsewhere in the specification. A preferred model is described in detail in the Experimental Results section.

Quantitative assessment of the extent of scarring (allowing generation of percentage values indicative of inhibition of scarring, as referred to throughout the specification) may be undertaken using any suitable protocol. It is preferred that such quantitative values are recorded using a suitable visual analogue scale (VAS). Suitable VAS may be used to assess scarring macroscopically or microscopically. Suitable criteria that may be considered in either macroscopic or microscopic assessment of scars are set out elsewhere in the specification. It may be preferred that assessment of scarring considers the macroscopic appearance of a scar, this being particularly important since it has a major impact on the extent of scarring perceived by the patient and by other observers.

A therapeutically effective amount of a GABA_{A} receptor agonist may preferably be an amount able to therapeutically alter the abundance and/or orientation of ECM components (such as collagen) in a treated scar.

A medicament of the invention will be capable of providing a therapeutically effective amount of a GABA_{A} receptor agonist, when administered in a suitable amount and via a suitable route. Preferably a medicament of the invention may be provided in the form of one or more dosage units. Each dosage unit may comprise a therapeutically effective amount of a GABA_{A} receptor agonist or a known fraction or multiple of such a therapeutically effective amount.

The inventors believe that the provision of between approximately 880 pg and 1 mg of a GABA_{A} receptor agonist, such as Gaboxadol, when provided in a single incidence of treatment per centimetre of a site that may otherwise be subject to scarring resulting from healing of a wound, may constitute a therapeutically effective amount in accordance with the present invention. Preferably a therapeutically effective amount of a GABA_{A} receptor agonist to be administered per centimetre in a single incidence of treatment, may be between about 880 pg and 9 μg, and more preferably between about 1.76 ng and 9 μg.

The inventors believe that GABA_{A} receptor agonist, such as Gaboxadol, may be provided in a therapeutically effective amount of between approximately 5 pmoles and 100 nmoles of the agonist, in a single incidence of treatment per centimetre of a site that may otherwise be subject to scarring. Preferably a therapeutically effective amount of a GABA_{A} receptor agonist to be administered per centimetre in a single incidence of treatment may be between about 5 pmoles and 50 nmoles, and more preferably between about 10 pmoles and 50 nmoles.

It may be preferred that a therapeutically effective amount of a GABA_{A} receptor agonist, such as Gaboxadol, considered in the preceding paragraphs be administered to a site requiring treatment twice over a period of approximately 24 hours. The inventors believe that these therapeutically effective amounts (i.e. between approximately 10 pmoles and 200 nmoles, preferably between 10 pmoles and 100 nmoles, and more preferably between about 20 pmoles and 100 nmoles) may also constitute preferred therapeutically effective amounts to be administered over the course of an entire regime of treatment (i.e. an effective treatment regime capable of preventing, reducing or inhibiting scarring to a desired extent may be achieved through just two incidences of administration in a period of approximately 24 hours).

Preferred therapeutically effective amounts of a GABA_{A} receptor agonist (either generally or with reference to particular selected agonists) may be investigated using in vitro and in vivo models, and suitable assessments of efficacy made with reference to various parameters for the measurement of scarring, as described elsewhere in the specification.

The skilled person will recognise that the information provided in the preceding paragraphs, as to amounts of a GABA_{A} receptor agonist that may be administered to inhibit scarring at a site that would otherwise be subject to scarring associated with healing of a wound, may be varied by the skilled practitioner in response to the specific clinical requirements of an individual patient. A physician (such as a physician with responsibility for the treatment of the patient in question) may determine suitable variations empirically, with reference to a range of factors including (but not limited to) the nature of the tissue to be treated, the area and/or depth of site to be treated, the severity of the wound, and the presence or absence of factors (such as infection) that may complicate healing or increase the likely magnitude of scarring, the nature of the scarring to be inhibited, and with reference to any inhibition of scarring already achieved.

In the event that a GABA_{A} receptor agonist is to be administered via topical administration, the amount provided may be altered depending on permeability of the tissue or organ to which the topical composition is administered. Thus, in the case of relatively impermeable tissues or organs, it may be preferred to increase the amount of the GABA_{A} receptor agonist to be administered. Such an increased amount of a GABA_{A} receptor agonist may still represent a therapeutically effective amount, if the amount of the agent taken up into the tissue or organ where scarring is to be inhibited is therapeutically effective.

It will be appreciated that the guidance provided herein, as to doses and amounts of an active agent to be used, is applicable to medicaments of the invention, and also to the methods of the invention.

The inventors have found that in one particularly preferred embodiment the GABA_{A} receptor agonist Gaboxadol may be administered in the form of a 1.76 ng/100 μl injectable solution, with 100 μl of such a solution provided per centimetre of a site to be treated over an approximately 24 hour period. The solution may preferably be provided by intradermal injection, and may be provided for two, or more, days.

In the case where the paragraphs above consider the administration of a specified amount of a medicament per linear cm of a wound it will be appreciated that this volume may be administered to either one or both of the margins of a wound to be treated (i.e. in the case of a reference to 100 μl of a medicament, this may be administered as 100 μl to either of the wound margins, or as 50 μl to each of the wound margins to be joined together). It may be preferred that the first incidence of treatment occurs prior to wounding, in which case the GABA_{A} receptor agonist may be provided to a site where a wound is to be formed. In the case that the GABA_{A} receptor agonist is provided by local injection to the skin (such as intradermal injection) this may cause a bleb to be raised as a result of the introduction of a solution containing the GABA_{A} receptor agonist into the skin. In one preferred embodiment the bleb may be raised in the site where the wound is to be formed, and indeed the wound may be formed by incising the bleb. In this case the amount of the GABA_{A} receptor agonist to be provided in the first incidence of treatment may be determined with reference to the length of the site where the wound is to be formed.

Alternatively two blebs may be raised, on either side of the site where the wound is to be formed. These blebs may preferably be positioned within half a centimetre of where the margins of the wound will be formed. In this case the amount of the GABA_{A} receptor agonist to be provided in the first incidence of treatment may be determined with reference to the length of the wound to be formed, measured in centimetres of future wound margin (defined below).

Preferably a bleb used to provide a GABA_{A} receptor agonist to a site prior to wounding may cover substantially the full length of the site where the wound is to be formed. More preferably the bleb may extend beyond the length of the site where a wound is to be formed. Suitably such a bleb may extend around half a centimetre (or more) beyond each end of the wound to be formed.

Intradermal injections in accordance with these embodiments of the invention may be administered by means of a hypodermic needle inserted substantially parallel to the midline of the wound to be formed, or parallel to the margins of the wound to be formed. Injection sites may be spaced approximately one centimetre apart from one another along the length of the region to which the GABA_{A} receptor agonist will be provided.

In the alternative, it may be preferred that the first incidence of treatment involves provision of a GABA_{A} receptor agonist to an existing wound. The inventors believe that the biological mechanisms relevant to the anti-scarring activity are the same whether cells are exposed to the GABA_{A} receptor agonist before or after wounding. In either case, the necessary biological activity may be achieved as long as the cells at the site where scarring is to be inhibited are exposed to a therapeutically effective amount of a GABA_{A} receptor agonist either before or after wounding.

### Centimetre of Wound

In the context of the present disclosure, a “centimetre of wound” represents a unit by which the size of site at which scarring is to be prevented, reduced or inhibited may be measured. For the present purposes, a centimetre of wound may be taken to encompass a site where a wound is to be formed, as well as a wounded site, or both margins of a wounded site (should such margins exist).

A centimetre of wound may be taken to comprise any square centimetre of a body surface that has, either in whole or in part, been wounded, or is to be wounded. For example, a wound of two centimetres length and one centimetre width (i.e. with a total surface area of two square centimetres) will be considered to constitute “two wound centimetres”, while a wound having a length of two centimetres and a width of two centimetres (i.e. a total surface area of four square centimetres) will constitute four wound centimetres. By the same token, a linear wound of two centimetres length, but of negligible width (i.e. with negligible surface area), will, for the purposes of the present invention, be considered to constitute “two wound centimetres”, if it passes through two square centimetres of the body surface.

The size of a site in wound centimetres should generally be assessed when the wound is in its relaxed state (i.e. when the body site bearing the site to be measured is in the position adopted when the body is at rest). In the case of the skin, the relevant size should be assessed when the skin is not subject to external tension.

### “Medicaments of the Invention”

For the purposes of the present disclosure, medicaments of the invention should be taken as encompassing any medicament manufactured in accordance with any aspect or embodiment of the invention, or any medicament comprising a GABA_{A} receptor agonist to be used in accordance with any aspect of the invention.

Medicaments of the invention will generally comprise a pharmaceutically acceptable excipient, diluent or carrier in addition to the GABA_{A} receptor agonist, such as Gaboxadol.

Medicaments of the invention may preferably be in the form of an injectable solution comprising a GABA_{A} receptor agonist. Solutions suitable for localised injection (and in particular for intradermal injection) constitute particularly preferred forms of the medicaments of the invention. For example, the inventors have found that medicaments comprising a GABA_{A} receptor agonist such as Gaboxadol dissolved in phosphate buffered saline may be administered by intradermal injection to reduce scarring resulting from healing of skin wounds.

### Preferred Body Sites

The inventors believe that GABA_{A} receptor agonists can be used in the prevention, reduction or inhibition of scarring resulting from healing of wound at any body site and in any tissue or organ. The skin represents a preferred site at which scarring may be prevented, reduced or inhibited utilising the medicaments or methods of the invention. Without wishing to limit the scope of the invention, the following passages provide guidance as to specific tissues and body sites that may benefit from inhibition of scarring using the medicaments or methods of the invention.

The use of a GABA_{A} receptor agonist to inhibit scarring formed on healing of a wound may bring about a notable improvement in the cosmetic appearance of an area thus treated. Cosmetic considerations are important in a number of clinical contexts, particularly when scars may be formed at prominent body sites such as the face, neck and hands. Consequently it is a preferred embodiment that the medicaments and methods of the invention be used to inhibit scarring at sites where it is desired to improve the cosmetic appearance of a scar formed.

In addition to its cosmetic impact, scarring of the skin is responsible for a number of deleterious effects afflicting those suffering from such scarring. For example, scarring of the skin may be associated with reduction of physical and mechanical function, particularly in the case of contractile scars and/or situations in which scars are formed across joints (articulations). The contraction exhibited by contractile scars of this kind is more pronounced than wound contraction that occurs as a normal part of the healing process, and may be distinguished from such normally occurring contraction in that it continues long after the healing process has ended (i.e. after wound closure). In cases of scars located in the area of joints, the altered mechanical properties of scarred skin, as opposed to unscarred skin, and the effects of scar contraction may lead to dramatically restricted movement of a joint so affected. Accordingly, it is a preferred embodiment that suitable medicaments and methods of the invention be used to inhibit scarring covering joints of the body. In another preferred embodiment suitable medicaments and methods of the invention may be used to inhibit scarring at increased risk of forming a contractile scar (in the case of scarring that results from the healing of wounds this may include wounds of children, and/or wounds produced by burns).

The extent of scar formation, and hence extent of cosmetic or other impairment that may be caused by the scar, may also be influenced by factors such as the tension of the site at which the scar or wound is formed. For example, it is known that skin under relatively high tension (such as that extending over the chest, or associated with lines of tension) may be prone to formation of more severe scars than at other body sites. Thus in a preferred embodiment suitable medicaments and methods of the invention may be used to inhibit scarring that results from the healing of wounds located at sites of high skin tension.

It will be appreciated that tissues other than the skin may also be subject to scarring resulting from the healing of wounds, and the medicaments and methods of the invention may also be of benefit in inhibiting scarring resulting from healing of wounds in these tissues.

The healing of wounds involving the peritoneum (the epithelial covering of the internal organs, and/or the interior of the body cavity) may frequently give rise to adhesions. Such adhesions are formed by bands of fibrous scar tissue, and can connect the loops of the intestines to each other, or the intestines to other abdominal organs, or the intestines to the abdominal wall. Adhesions can pull sections of the intestines out of place and may block passage of food. Adhesions are also a common sequitur of surgery involving gynaecological tissues. Incidences of adhesion formation may be increased in wounds that are subject to infection (such as bacterial infection) or exposure to radiation. The medicaments or methods of the invention may be beneficial in inhibiting scarring resulting from the healing of wounds that may otherwise lead to adhesion formation.

The medicaments or methods of the invention are suitable for use in the inhibition of scarring that results from healing of wounds of the eye (and particularly of the cornea or retina), and their use in this context represents a preferred embodiment of the invention. Merely by way of example, the medicaments or methods of the invention may be used to inhibit scarring associated with accidental injury or as a result of surgical operations such as glaucoma filtration surgery (such as at sites of pressure relieving blebs formed during such surgery) or corneal surgery (such as photorefractive keratectomy—PRK; laser epithelial keratomileusis—LASEK; or laser assisted in situ keratomileusis—LASIK) or cataract surgery (where scarring may frequently be associated with contraction of the lens capsule).

Scarring in blood vessels, e.g. following anastomotic surgery, can lead to myointimal hyperplasia and reduction in the volume of the blood vessel lumen (restenosis). A therapeutically effective amount of a GABA_{A} receptor agonist may be provided to blood vessels in order to inhibit such scarring.

The medicaments or methods of the invention may be used to inhibit scarring in tendons and ligaments. Such scarring may otherwise be expected to occur following surgery or trauma involving tissues of this type.

### Preferred Wounds

The inventors believe that GABA_{A} receptor agonists may be used in medicaments or methods of the invention to beneficially inhibit scar formation resulting from all types of wounds.

Examples of specific wounds in which scarring may be inhibited using the medicaments and methods of the invention include, but are not limited to, wounds independently selected from the group consisting of: wounds of the skin; wounds of the eye (including the inhibition of scarring resulting from eye surgery such as LASIK surgery, LASEK surgery, PRK surgery, glaucoma filtration surgery, cataract surgery, or surgery in which the lens capsule may be subject to scarring) such as those giving rise to corneal cicatrisation; wounds subject to capsular contraction (which is common surrounding breast implants); wounds of blood vessels; wounds of tendons, ligaments or muscle; wounds of the oral cavity, including the lips and palate (for example, to inhibit scarring resulting from treatment of cleft lip or palate); wounds of the internal organs such as the liver, heart, brain, digestive tissues and reproductive tissues; wounds of body cavities such as the abdominal cavity, pelvic cavity and thoracic cavity (where inhibition of scarring may reduce the number of incidences of adhesion formation and/or the size of adhesions formed); and surgical wounds (in particular wounds associated with cosmetic procedures, such as scar revision). It is particularly preferred that the medicaments and methods of the invention be used to prevent, reduce or inhibit scarring associated with wounds of the skin.

The inventors believe that the ability of the medicaments and methods of the invention to inhibit scarring may reduce the occurrence of adhesions (such as those occurring in the abdomen, pelvis, thorax or spine). Accordingly, the use of medicaments or methods of the invention employing a GABA_{A} receptor agonist to prevent the formation of adhesions represents a preferred embodiment of the invention. The use of medicaments or methods of the invention in the inhibition of scarring resulting from wounds of the peritoneum is another preferred embodiment.

The medicaments and methods of the invention may be useful in the inhibition of scarring resulting from healing of infected wounds or wounds exposed to radiation.

Incisional wounds are a preferred group of wounds scarring resulting from which may be inhibited by the medicaments and methods of the invention. Surgical incisional wounds may constitute a particularly preferred group of wounds in respect of which scarring may be inhibited utilising the medicaments and methods of the invention.

It is a preferred embodiment that the medicaments and methods of the invention be used to inhibit scarring associated with plastic or cosmetic surgery. Since a large number of plastic or cosmetic surgeries consist of elective surgical procedures it is readily possible to administer a GABA_{A} receptor agonist, such as Gaboxadol, prior to surgery, and/or around the time of closure of the wound (for instance, before or after the application of sutures), and this use represents a particularly preferred embodiment of the invention.

In surgical procedures in general, a preferred route by which a GABA_{A} receptor agonist may be administered is via localised injection (such as intradermal injection). Such injections may form raised blebs, which may then be incised as part of the surgical procedure, or alternatively the bleb may be raised by injecting the wound margins after the wound has been closed e.g. by sutures.

Scar revisions are surgical procedures in which existing scars are “revised” (for example through excision or realignment) in order to reduce the cosmetic and/or mechanical disruption caused by the existing scar. Probably the best known of these is “Z-plasty” in which two V-shaped flaps of skin are transposed to allow rotation of a line of tension. The use of the medicaments or methods of the invention in procedures associated with scar revision represents a preferred use in accordance with the present invention.

It may generally be preferred that the medicaments or methods of the invention, in which a GABA_{A} receptor agonist, such as Gaboxadol, is used to inhibit scarring, be used to inhibit scarring that occurs as part of the “normal” healing response. Thus the medicaments or methods of the invention may be used in the treatment of normal (as opposed to chronic) wounds and for the inhibition of non-pathological scarring (e.g. scarring occurring in a patient without a history of, or susceptibility to, keloids, hypertrophic scars, pterygium, or the like). A history of pathological scarring, or susceptibility to pathological scarring, may be identified with reference to a patient's clinical history, or by means of tests for genetic markers known to be associated with a predisposition to pathological scarring.

It is often clinically necessary to surgically revise hypertrophic scars or keloids, the aim of such revision being to replace the relatively severe pathological scar with a less noticeable non-pathological scar. Revision in this manner may be designed to reduce incidence of pathological scarring by, for example, reducing tension at the site of the revised scar, or by taking other appropriate steps to inhibit scarring. Thus, it is a further preferred embodiment of the invention that the medicaments or methods herein described be used to inhibit non-pathological scarring that results from wounds produced during surgical revision of pathological scars.

It is recognised that wounds resulting from burns injuries (which for the purposes of the present invention may be taken to encompass exposure to heated gasses or solids, as well as scalding injuries involving hot liquids; “freezer burn” injuries caused by exposure to extreme low temperatures; radiation burns; and chemical burns, such as those caused by caustic agents) may extend over great areas of an individual so afflicted. Accordingly, burns may give rise to scar formation covering a large proportion of a patient's body. This great extent of coverage increases the risk that the scar formed will cover areas of elevated cosmetic importance (such as the face, neck, arms or hands) or of mechanical importance (particularly the regions covering or surrounding joints). Burns injuries caused by hot liquids are frequently suffered by children (for example as a result of upsetting pans, kettles or the like) and, due to the relatively smaller body size of children, are particularly likely to cause extensive damage over a high proportion of the body area. Thus there is an elevated risk of both cosmetic and mechanical impairment associated with scarring after burns. Accordingly, it is a preferred embodiment that medicaments and methods of the invention be used to inhibit scarring resulting from burns injuries.

The ability of a GABA_{A} receptor agonist, such as Gaboxadol, to inhibit scarring is of great utility in the inhibition of scarring associated with grafting procedures. In particular, the medicaments and methods of the invention may be used to inhibit scarring that results from wounds associated with grafting procedures. Inhibition of scarring using the medicaments and methods of the invention is of benefit both at a graft donor sites and graft recipient sites. The scar inhibitory effects of the medicaments and methods of the invention are able to inhibit scarring that may otherwise occur at sites where tissue for grafting is removed, or that may be associated with the healing and integration of grafted tissue. The inventors believe that the methods and medicaments of the invention confer advantages in the inhibition of scarring that may otherwise be associated with grafts utilising skin, artificial skin, or skin substitutes.

The inventors also believe that the medicaments and methods of the invention may be used to inhibit scarring associated with encapsulation. Encapsulation is a form of scarring that occurs around sites at which implant materials (such as biomaterials) have been introduced into the body. Encapsulation is a frequent complication associated with breast implants, and the use of a GABA_{A} receptor agonist, such as Gaboxadol, to inhibit encapsulation in this context is a preferred embodiment of the invention.

The medicaments and methods of the invention may be used to inhibit scarring that results from healing of wounds selected from the group consisting of: abrasions (also commonly referred to as “scrapes”, these are shallow injuries which frequently cover a relatively large area); avulsions (when an entire bodily structure, or a part of such a structure, is forcibly pulled away from its site); crush wounds; incisional wounds; lacerations; punctures; and missile wounds. All of these different types of wounds may be suffered by the skin, among other tissues or organs, and all may, to a greater or lesser extent, result in scarring.

The wounds resulting from surgical procedures are most commonly incisional wounds, and these are a frequent cause of scarring. Accordingly it is a preferred embodiment that the medicaments and methods of the invention be used in the inhibition of scarring resulting from incisional wounds, such as surgical wounds. It is believed that each year 84 million surgical procedures are conducted worldwide in which the skin is incised. Accordingly, it can be seen that the potential market for, and potential benefits provided by, the medicaments and methods of the invention are very large indeed.

The inventors believe that the medicaments or methods of the invention may be of use in inhibiting scarring associated with full thickness or partial thickness wounds (respectively wounds in which the epithelial layer is either totally or partly compromised). Preferred examples of partial thickness wounds, scarring associated with which may be inhibited using the medicaments or methods of the invention, include “skin peels” such as “chemical peels” (such as alphahydroxy acid peels, trichloroacetic acid peels or phenol peels) or “laser peels”; wounds associated with dermabrasion; and wounds associated with dermaplaning. It may particularly be preferred that the medicaments or methods of the invention be used to inhibit scarring associated with partial thickness wounds occurring at cosmetically important sites (such as the face), which may frequently be the subject of skin peel treatment.

### Prevention, Reduction or Inhibition of Scarring

The prevention, reduction or inhibition of scarring within the context of the present invention should be understood to encompass any degree of prevention, reduction or inhibition in scarring achieved on healing of a treated wound, as compared to the level of scarring occurring on healing of a control-treated or untreated wound. Throughout the specification references to “prevention”, “reduction” or “inhibition” of scarring are generally to be taken, except where the context requires otherwise, to represent substantially equivalent activities, involving equivalent mechanisms mediated by GABA_{A} receptor agonists, such as Gaboxadol.

For the sake of brevity, the present specification will primarily refer to “inhibition” of scarring utilising a GABA_{A} receptor agonist, such as Gaboxadol. However, such references should be taken, except where the context requires otherwise, to also encompass the prevention or reduction of scarring using such compounds.

The inhibition of scarring achieved using methods and medicaments of the invention may be assessed and/or measured with reference to the microscopic and/or macroscopic appearance of a treated scar. Inhibition of scarring may also suitably be assessed by comparing the microscopic and/or macroscopic appearance of a treated scar with the microscopic and/or macroscopic appearance of an untreated scar. In general, it may be preferred that inhibition of scarring using medicaments or methods utilising a GABA_{A} receptor agonist, such as Gaboxadol, are assessed with reference to the macroscopic appearance of a treated scar, since the macroscopic appearance of the scar may most accurately reflect the way in which the scar is perceived either by the scarred subject, or by others.

Suitable methods and parameters by which scarring may be assessed in treated scars or control scars (and hence any inhibition of scarring identified) are described elsewhere in the specification, as are methods by which such assessments may be captured and quantified (if so required).

In the event that scarring is assessed quantitatively, it may be preferred that inhibition of scarring be indicated by a statistically significant decrease in a treated wound or scar, as compared to a control counterpart.

### “Treated Wounds”, “Untreated Wounds”, “Treated Scars” and “Untreated Scars”

Treatment of wounds with a therapeutically effective amount of a GABA_{A} receptor agonist, such as Gaboxadol, will inhibit the scarring that may otherwise be expected to occur on the healing of untreated wounds.

For present purposes an “untreated wound” should be considered to be any wound that has not been exposed to a therapeutically effective amount of a GABA_{A} receptor agonist, such as Gaboxadol. A “diluent control-treated wound” will be an untreated wound to which a control diluent has been administered, and a “naïve control” will be an untreated wound made without administration of a GABA_{A} receptor agonist, such as Gaboxadol, and without a suitable control diluent, and left to heal without therapeutic intervention.

In contrast, a “treated wound” may be considered to be a wound exposed to a therapeutically effective amount of a GABA_{A} receptor agonist, such as Gaboxadol. Thus a treated wound may be a wound which has been provided with a medicament of the invention, or which has received treatment in accordance with the methods of the invention.

For the present purposes a “treated scar” should be taken as comprising a scar that results from healing of a treated wound (i.e. a wound treated with a therapeutically effective amount of a GABA_{A} receptor agonist, such as Gaboxadol).

By way of contrast, an “untreated scar” should be taken as comprising a scar that results from healing of an untreated wound (for example a wound treated with a placebo, control, or standard care).

Untreated scars may typically be used as comparators in assessing the inhibition of scarring that may be evident in a treated scar. Suitable comparator untreated scars of this type may preferably be matched to the treated scar with reference to one or more criteria independently selected from the group consisting of: scar age; scar size; scar site; Body Mass Index of patients; patient age; patient race and patient gender.

### Models of Scarring

In the case of inhibition of scarring that results from the healing of a wound, a suitable animal model in which the therapeutic effectiveness of a GABA_{A} receptor agonist, such as Gaboxadol, may be assessed, and in which a therapeutically effective amount of an active agent may be determined, may involve providing the GABA_{A} receptor agonist, such as Gaboxadol, to incisional or excisional wounds of experimental subjects (either humans subjects, or non-human animals such as mice, rats or pigs), and assessing the scarring that results on healing of the wound. Suitable models may utilise full thickness or partial thickness wounds depending on the wounding that it is intended to treat. Examples of models of full or partial thickness wound healing are well known to those skilled in the art.

The experimental models described above may also allow identification of particular effective routes or regimes by which a GABA_{A} receptor agonist, such as Gaboxadol, may be administered. These routes or regimes may provide notable advantages in the context of the medicaments and methods of the present invention, and these may give rise to further aspects of the invention.

### Assessment of Scarring, and of Inhibition of Scarring

The extent of inhibition of scarring that may be required in order to achieve a therapeutic effect will be apparent to, and may readily be determined by, a clinician responsible for the care of the patient. The clinician may undertake a suitable determination of the extent of inhibition of scarring that has been achieved using a GABA_{A} receptor agonist, such as Gaboxadol, in order to assess whether or not a therapeutic effect has been achieved, or is being achieved. Such an assessment may, but need not necessarily, be made with reference to suggested methods of measurement described herein.

The extent to which inhibition of scarring utilising a GABA_{A} receptor agonist, such as Gaboxadol, is achieved may be assessed with reference to the effects that such an active agent may achieve in human patients treated with the methods or medicaments of the invention. Alternatively, inhibition of scarring that may be achieved by a GABA_{A} receptor agonist, such as Gaboxadol, may be assessed with reference to experimental investigations using suitable in vitro or in vivo models. The use of experimental models to investigate inhibition of scarring may be particularly preferred in assessing the therapeutic effectiveness of particular GABA_{A} receptor agonists, or in establishing therapeutically effective amounts of such agonists, such as Gaboxadol.

Animal models of wound healing and scar formation represent preferred experimental models for in vivo assessment of the extent of scar inhibition that may be achieved using the medicaments or methods of the invention. Examples of such models are described below for illustrative purposes. The models of scarring and methods for assessing scarring described herein may be used to determine therapeutically effective GABA_{A} receptor agonists, and therapeutically effective amounts of agonists.

Inhibition of scarring, using the medicaments and methods of the invention, can be effected at any body site and in any tissue or organ so far investigated. For illustrative purposes the scar inhibitory activity of medicaments and methods of the invention will primarily be described with reference to inhibition of scarring that may be brought about in the skin (the body's largest organ). However, the skilled person will immediately appreciate that many of the factors that are relevant when considering inhibition of scarring in the skin are also relevant to inhibition of scarring in other organs or tissues. Accordingly the skilled person will recognise that, except for where the context requires otherwise, the parameters and assessments considered below in respect of scars of the skin may also be applicable to scarring in tissues other than the skin.

In the skin, treatment may improve the macroscopic and microscopic appearance of scars; macroscopically the scars may be less visible and blend with the surrounding skin, microscopically the collagen fibres within the scar may have morphology and organisation that is more similar to those in the surrounding skin.

The inhibition of scarring achieved using methods and medicaments of the invention may be assessed and/or measured with reference to either the microscopic or macroscopic appearance of a treated scar as compared to the appearance of an untreated scar. Inhibition of scarring may also suitably be assessed with reference to both macroscopic and microscopic appearance of a treated scar.

In considering the macroscopic appearance of a scar resulting from a treated wound, the extent of scarring, and hence the magnitude of any inhibition of scarring achieved, may be assessed with reference to any of a number of parameters. Most preferably, holistic assessment of the scar by means of assessment of macroscopic photographs by an independent expert panel, by means of an independent lay panel or clinically by means of a macroscopic assessment by a clinician of the patients themselves. Assessments are captured by means of a VAS (visual analogue scale) or a categorical scale.

Macroscopic characteristics of a scar which can be assessed objectively include:
i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Similarly, scars may be redder than the surrounding skin. In this case inhibition of scarring may be demonstrated when the redness of a treated scar fades earlier, or more completely, or to resemble more closely the appearance of the surrounding skin, compared to an untreated scar. There are a number of non-invasive colorimetric devices which are able to provide data with respect to pigmentation of scars and unscarred skin, as well as redness of the skin (which may be an indicator of the degree of vascularity present in the scar or skin). Examples of such devices include the X-rite SP-62 spectrophotometer, Minolta Chronometer CR-200/300; Labscan 600; Dr. Lange Micro Colour; Derma Spectrometer; laser-Doppler flow meter; and Spectrophotometric intracutaneous Analysis (SIA) scope.
ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar. Height of the scar can be measured directly on a patient by means of profilometry, or indirectly, by profilometry of moulds taken from a scar.
iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can be measured directly on a patient by means of profilometry, or indirectly by profilometry of moulds taken from a scar.
iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two parameters, even more preferably at least three parameters, and most preferably at least four of these parameters (for example, all four of the parameters set out above). The parameters described above may be used in the development of a visual analogue scale (VAS) for the macroscopic assessment of scarring. Details regarding implementation of VASs are described below.

Microscopic assessment may also provide a suitable means by which the quality of treated and untreated or control scars may be compared. Microscopic assessment of scar quality may typically be carried out using histological sections of scars. Suitable parameters for the microscopic assessment of scars may include:
i) Thickness of extracellular matrix (ECM) fibres. Scars typically contain thinner ECM fibres than are found in the surrounding skin. This property is even more pronounced in the case of keloid and hypertrophic scars, and this may provide one of the histological markers by which such scars can be differentiated from “normal” scarring of the type addressed by the invention. Inhibition of scarring may be demonstrated when the thickness of ECM fibres in a treated scar more closely approximates the thickness of ECM fibres found in unscarred skin than does the thickness of fibres found in an untreated scar.
ii) Orientation of ECM fibres. ECM fibres found in scars tend to exhibit a greater degree of alignment with one another than do those found in unscarred skin (which have a random orientation frequently referred to as “basket weave”). The ECM of pathological scars such as keloids and hypertrophic scars may exhibit even more anomalous orientations, frequently forming large “swirls” or “capsules” of ECM molecules. Accordingly, inhibition of scarring may be demonstrated when the orientation of ECM fibres in a treated scar more closely approximates the orientation of ECM fibres found in unscarred skin than does the orientation of such fibres found in an untreated scar.
iii) ECM composition of the scar. The composition of ECM molecules present in scars shows differences from that found in normal skin, with a reduction in the amount of elastin present in ECM of scars. Thus inhibition of scarring may be demonstrated when the composition of ECM fibres in the dermis of a treated scar more closely approximates the composition of such fibres found in unscarred skin than does the composition found in an untreated scar.
iv) Cellularity of the scar. Scars tend to contain relatively fewer cells than does unscarred skin. It will therefore be appreciated that inhibition of scarring may be demonstrated when the cellularity of a treated scar more closely approximates the cellularity of unscarred skin than does the cellularity of an untreated scar.

Other features that may be taken into account in assessing the microscopic quality of scars include elevation or depression of the scar relative to the surrounding unscarred skin, and the prominence or visibility of the scar at the interface with the unscarred skin

The parameters described above may be used in generating a VAS for the microscopic assessment of scarring. Such a VAS may consider collagen organisation and abundance in the papillary dermis and the reticular dermis may also provide a useful index of scar quality. Inhibition of scarring may be indicated when the quality of a treated scar is closer to that of unscarred skin than is the quality of an untreated or control scar.

It is surprising to note that the overall appearance of scars, such as those of the skin, is little influenced by the epidermal covering of the scar, even though this is the part of the scar that is seen by the observer. Instead, the inventors find that the properties of the connective tissue (such as that making up the dermis, or neo-dermis) present within the scar have greater impact on the perception of extent of scarring, as well as on the function of the scarred tissue. Accordingly assessments of criteria associated with the connective tissues such as the dermis, rather than epidermis, may prove to be the most useful in determining inhibition of scarring.

The thickness of ECM fibres and orientation of ECM fibres may be favoured parameters, for assessing inhibition of scarring. A treated scar may preferably have improved ECM orientation (i.e. orientation that is more similar to unscarred skin than is the orientation in an untreated scar).

A treated scar will preferably demonstrate inhibition of scarring as assessed with reference to at least one of the parameters for microscopic assessment set out above. More preferably a treated scar may demonstrate inhibition of scarring with reference to at least two of the parameters, even more preferably at least three of the parameters, and most preferably all four of these parameters.

It will be appreciated that inhibition of scarring achieved using the medicaments or methods of the invention may be indicated by improvement of one or more suitable parameters combined from different assessment schemes (e.g. inhibition as assessed with reference to at least one parameter used in macroscopic assessment and at least one parameter used in microscopic assessment).

Further examples of suitable parameters for the clinical measurement and assessment of scars may be selected based upon a variety of measures or assessments including those described by Duncan et al. (2006), Beausang et al. (1998) and van Zuijlen et al. (2002). Except for where the context requires otherwise, many of the following parameters may be applied to macroscopic and/or microscopic assessment of scarring. Examples of Suitable parameters for assessment of scars in the skin may include:

1. Assessment with Regard to Visual Analogue Scale (VAS) Scar Score.

Prevention, reduction or inhibition of scarring may be demonstrated by a reduction in the VAS score of a treated scar when compared to a control scar. A suitable VAS for use in the assessment of scars may be based upon the method described by Duncan et al. (2006) or by Beausang et al. (1998). This is typically a 10 cm line in which 0 cm is considered an imperceptible scar and 10 cm a very poor hypertrophic scar.

2. Assessment with Regard to a Categorical Scale.

Prevention, reduction or inhibition of scarring may be determined by allocating scars to different categories based on either textual descriptions e.g. “barely noticeable”, “blends well with normal skin”, “distinct from normal skin”, etc., by comparing a treated scar and a an untreated or control scar, noting any differences between these, and allocating the differences to selected categories (suitable examples of which may be “mild difference”, “moderate difference”, “major difference”, etc.). Assessment of this sort may be performed by the patient, by an investigator, by an independent panel, or by a clinician, and may be performed either directly on the patient or on photographs or moulds taken from the patient. Inhibition of scarring may be demonstrated when an assessment indicates that treated scars are generally allocated to more favourable categories than are untreated or control scars.

### 3. Scar Height, Scar Width, Scar Perimeter, Scar Area or Scar Volume.

The height and width of scars can be measured directly upon the subject, for example by use of manual measuring devices such as callipers, or automatically with the use of profilometers. Scar width, perimeter and area may be measured either directly on the subject, by image analysis of photographs of the scar, by analysis of silicone mould impressions of the scar, or by analysis of positive casts made from such impressions. The skilled person will also be aware of further non-invasive methods and devices that can be used to investigate suitable parameters, including silicone moulding, ultrasound, optical three-dimensional profilimetry and high resolution Magnetic Resonance Imaging.

Inhibition of scarring may be demonstrated by a reduction in the height, width, area, perimeter or volume, or any combination thereof, of a treated scar as compared to an untreated scar.

### 4. Scar Distortion and Mechanical Performance

Scar distortion may be assessed by visual comparison of a scar and unscarred skin. A suitable comparison may categorise a selected scar as causing no distortion, mild distortion, moderate distortion or severe distortion.

The mechanical performance of scars can be assessed using a number of non-invasive methods and devices based upon suction, pressure, torsion, tension and acoustics. Suitable examples of devices capable of use in assessing mechanical performance of scars include Indentometer, Cutometer, Reviscometer, Visco-elastic skin analysis, Dermaflex, Durometer, Dermal Torque Meter and Elastometer.

Inhibition of scarring may be demonstrated by a reduction in distortion caused by treated scars as compared to that caused by untreated scars. It will also be appreciated that inhibition of scarring may be demonstrated by the mechanical performance of unscarred skin being more similar to that of treated scars than of untreated scars.

### Photographic Assessments

### Independent Lay Panel

Photographic assessment of treated and untreated scars may be performed by an independent lay panel of assessors using standardised and calibrated photographs of the scars. The scars may be assessed by an independent lay panel to provide categorical ranking data (e.g. that a given treated scar is “better”, “worse” or “no different” when compared to an untreated scar) and quantitative data using a Visual Analogue Scale (VAS) based upon the method described by Duncan et al. (2006) and Beausang et al. (1998). The capture of these data may make use of suitable software and/or electronic system(s) as described in the applicant's co-pending patent application filed as PCT/GB2005/004787.

### Expert Panel

Photographic assessment of treated and untreated scars may alternatively or additionally be performed by a panel of expert assessors using standardised and calibrated photographs of the scars to be assessed, and/or positive casts of silicone moulds. The panel of experts may preferably consist of individuals skilled in the art, suitable examples of which include plastic surgeons, dermatologists or scientists having relevant technical backgrounds.

### Clinical Assessment

A clinician, or an independent panel of clinicians may assess the scar(s) on a patient using any of the forgoing parameters e.g. VAS, colour, categorical scales, etc. A suitable clinician may be a clinician responsible for care of a patient, or may be a clinician investigating efficacy of therapies for inhibition of scarring.

### Patient Assessment

A patient may assess their own scars and/or compare scars by means of a structured questionnaire. A suitable questionnaire may measure parameters such as: the patient's satisfaction with their scar; how well the scar blends with the unscarred skin; as well as the effect of the scar on their daily life (suitable questions may consider whether the patient uses clothes to hide the scar, or otherwise avoids exposing it) and/or scar symptoms (examples of which may include itch, pain or paresthesia) Inhibition of scarring may be indicated by the treated scar receiving a more positive rating from the patient, and/or causing the patient fewer problems, and/or causing fewer or less scar symptoms, and/or an increase in patient satisfaction compared to an untreated scar.

In addition to categorical data, quantitative data (preferably relating to the above parameters) can be generated using image analysis in combination with suitable visualisation techniques. Examples of suitable visualisation techniques that may be employed in assessing scar quality are specific histological stains or immuno-labelling, wherein the degree of staining or labelling present may be quantitatively determined by image analysis

Quantitative data may be usefully and readily produced in relation to the following parameters:

1. Scar width, height, elevation, volume and area.
2. Collagen organisation, collagen fibre thickness, collagen fibre density.
2. Number and orientation of fibroblasts.
4. Quantity and orientation of other ECM molecules e.g. elastin, fibronectin

Prevention, reduction or inhibition of scarring may be demonstrated by a change in any of the parameters considered above such that a treated scar more closely resembles unscarred skin than does a control or untreated scar (or other suitable comparator).

The assessments and parameters discussed above are suitable for assessment of the effects of a GABA_{A} receptor agonist, such as Gaboxadol, on scar formation, as compared to control, placebo or standard care treatment in animals or humans. It will be appreciated that these assessments and parameters may be utilised in determining a therapeutically effective GABA_{A} receptor agonist that may be used for scar prevention, reduction or inhibition; and in determining therapeutically effective amounts of GABA_{A} receptor agonists, such as Gaboxadol. Appropriate statistical tests may be used to analyse data sets generated from different treatments in order to investigate the significance of results.

Other parameters that may be used in the assessment of scarring in organs other than the skin may be determined with reference to the organ in question. For example, corneal scarring may be assessed by measuring the opacity, or transmitting/refractory properties, of the cornea and measurement of corneal curvature. Such assessments may, for example, be made using in vivo confocal microscopy and/or specular microscopy or corneal topography

Successful inhibition of scarring in tendons or ligaments may be indicated by restoration of function of tissues treated with the medicaments or methods of the invention. Suitable indicators of function may include the ability of the tendon or ligament to bear weight, stretch, flex, etc. Such assessments may, for example, be made using electrophysiological reflex examination, surface electromyography, ultrasonography, ultrasound/MRI scan, and self reported symptom and pain questionnaires

The extent of scarring occurring in blood vessels can be measured directly e.g. using ultrasound, or indirectly by means of blood flow. Inhibition of scarring achieved using the medicaments or methods of the invention may lead to a reduction in narrowing of the blood vessel lumen and allow a more normal blood flow.

### Administration Regimes

The medicaments of the invention should be provided to the site where there therapeutic activity is to be required before the healing response has been completed yielding a scar. The methods or medicaments of the invention may be used to provide a therapeutically effective amount of a GABA_{A} receptor agonist, such as Gaboxadol, to a site of an existing wound. Alternatively, the medicaments or methods of the invention may be used prophylactically, i.e. prior to wound formation.

In the case of the inhibition of scarring associated with healing of a wound, prophylactic use may involve administration of a therapeutically effective amount of a GABA_{A} receptor agonist, such as Gaboxadol, at sites where no wound presently exists, but where a wound that would otherwise give rise to a scar is to be formed. By way of example, a therapeutically effective amount of a GABA_{A} receptor agonist, such as Gaboxadol, may be administered to sites that are to undergo wounding as a result of elective procedures (such as surgery), or to sites that are believed to be at elevated risk of wounding.

It may be preferred that the medicaments of the invention are administered to the site around the time of wounding, or immediately prior to the forming of a wound (for example in the period up to six hours before wounding) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may determined with reference to factors such as the patient's age, health, and predisposition to healing complications or adverse scarring). The prophylactic use of methods and medicaments in accordance with the invention is a preferred embodiment of the invention, and is particularly preferred in the prevention, reduction or inhibition of scarring in the context of surgical wounds.

The methods and medicaments of the invention are also able to inhibit scarring if administered after a wound has already been formed. It is preferred that such administration should occur as early as possible after formation of the wound, but agents of the invention are able to inhibit scarring at any time up until the healing process has been completed (i.e. even in the event that a wound has already partially healed the methods and medicaments of the invention may be used to inhibit scarring in respect of the remaining un-healed portion). It will be appreciated that the “window” in which the methods and medicaments of the invention may be used to inhibit scarring is dependent on the nature of the wound in question (including the degree of damage that has occurred, and the size of the wounded area). Thus, in the case of a large wound, the methods and medicaments of the invention may be administered relatively late in the healing response yet still be able to inhibit scarring, as a consequence of the relatively prolonged time that large wounds require to heal.

The methods and medicaments of the invention may, for instance, preferably be administered within the first 24 hours after a wound is formed, but may still inhibit scarring if administered up to ten, or more, days after wounding.

The methods and medicaments of the invention may be utilised after a wound has been closed (e.g. by suturing) to inhibit scarring that would otherwise be associated with healing of the wound. Provision of a GABA_{A} receptor agonist such as Gaboxadol to a wound may be repeated as often as necessary after wound closure in order to bring about a desired inhibition in scarring.

The methods and medicaments of the invention may be administered on one or more occasions (as necessary) in order to inhibit scarring. Suitable regimes may involve administration monthly, weekly, daily or twice daily.

For instance, in the case of inhibition of scarring that results from the healing of a wound, therapeutically effective amounts of a GABA_{A} receptor agonist, such as Gaboxadol, may be administered to a wound as often as required until the healing process has been completed. By way of example, the medicaments of the invention may be administered daily or twice daily to a wound for at least the first three days following the formation of the wound. In a particularly preferred embodiment a medicament of the invention may be administered prior to wounding and again approximately 24 hours following wounding.

Most preferably the methods or medicaments of the invention may be administered both before and after formation of a wound. The inventors have found that administration of the medicaments of the invention immediately prior to the formation of a wound, followed by daily administration of a GABA_{A} receptor agonist, such as Gaboxadol, for one or more days following wounding, is particularly effective in inhibiting scarring resulting from the healing of a wound.

A therapeutically effective amount of a GABA_{A} receptor agonist, such as Gaboxadol, may be provided by means of any number of suitable administrations. Suitable regimes for these administrations may be readily devised by the skilled person using techniques (including in vitro studies, animal and human studies) well known and established within the pharmaceutical industry.

It will be appreciated that the amount of a medicament of the invention that should be provided to a wound, in order that a therapeutically effective amount of an active agent may be administered, depends on a number of factors. These include the biological activity and bioavailability of the agent present in the medicament, which in turn depends, among other factors, on the nature of the agent and the mode of administration of the medicament. Other factors in determining a suitable therapeutic amount of a medicament may include:
A) The half-life of the active agent in the subject being treated.
B) The specific condition to be treated (e.g. incisional or excisional wounding).
C) The age of the subject.
D) The size of the site to be treated.

The frequency of administration will also be influenced by the above-mentioned factors and particularly the half-life of the chosen agent within the subject being treated.

Generally when medicaments in accordance with the invention are used to treat existing wounds the medicament should be administered as early as possible in the healing process. In the case of wounds that are not immediately apparent, such as those at internal body sites, medicaments may be administered as soon as the wound, and hence the risk of scarring, is diagnosed. Therapy with methods or medicaments in accordance with the invention should continue until scarring has been inhibited to a clinician's satisfaction.

Frequency of administration will depend upon the biological half-life of the agent used. Typically a cream or ointment containing an agent of the invention should be administered to a target tissue such that the concentration of the agent at a wound or scar is maintained at a level suitable to inhibit or reduce scarring. This may require administration daily or even several times daily. The inventors have found that administration of an agent of the invention immediately prior to wounding, with a further administration one day after wounding is particularly effective for the inhibition of scarring that would otherwise result from the healing of such a wound.

Daily doses of an agent of the invention may be given as a single administration (e.g. a daily application of a topical formulation or a daily injection). Alternatively, the agent of the invention may require administration twice or more times during a day. In a further alternative, a slow release device may be used to provide optimal doses of an agent of the invention to a patient without the need to administer repeated doses.

### Routes of Administration

Any suitable route capable of achieving the desired effect of inhibiting scarring may be used to administer a therapeutically effective amount of a GABA_{A} receptor agonist. However, it may generally be preferred that the GABA_{A} receptor agonist is provided to a tissue, the scarring of which is to be inhibited, by local administration.

Suitable methods by which such local administration may be achieved will depend on the identity of the tissue or organ in question. The selection of preferred routes of administration may also depend on whether or not a tissue or organ to be treated is permeable to the chosen medicament. Suitable routes of administration may be selected from the group consisting of: injections; application of sprays, ointments, gels or creams; inhalation of medicaments; release from biomaterials or other solid medicaments including sutures or wound dressings. Generally, preferred routes of administration may include local injection (for example intradermal injection in the case where it is wished to inhibit scarring of the skin). Suitable formulations for use in these embodiments of the invention are considered elsewhere in the specification.

Medicaments of the invention may be administered in a topical form to inhibit scarring formed on healing of a wound. Suitably, such administration may be effected as part of the initial and/or follow up care for the wounded area. Injections may be administered around the margins of a wound. In the case of their prophylactic use, medicaments of the invention may be applied to a site where a wound will occur.

Preferred routes of administration may be selected with reference to the tissue or organ to be treated. In the case of corneal scarring, medicaments of the invention may be administered to the outer surfaces of the eye, such as the cornea. Application of the medicament may be by means of local eye drops (including viscous or semi-viscous eye drops), creams, gels, ointments, or the like, and may, for example be applied using an eye dropper or sponge applicator.

In the case where it is wished to provide a therapeutically effective amount of a GABA_{A} receptor agonist, such as Gaboxadol, to internal wounds such as those caused by surgical procedures (which may otherwise be prone to formation of adhesions), medicaments comprising a GABA_{A} receptor agonist may be administered by lavage, or by means of a parenteral gel/instillate, or by means of release from local devices (such as sutures, films or carriers able to release GABA_{A} receptor agonists to their surroundings) that may be inserted at the time of surgery.

In the case of scarring of blood vessels, suitable routes of administration may include direct injection into the walls of the blood vessel (for instance before suturing), bathing an anastomotic site in a medium comprising a GABA_{A} receptor agonist, such as Gaboxadol, or administration of an agonist by local applied devices, e.g. sutures or stents. Effective inhibition of scarring in blood vessels may be indicated by the maintenance of a normal level of blood flow following blood vessel injury.

In the case where it is wished to inhibit scarring occurring in a relatively inaccessible body site it may be preferred that GABA_{A} receptor agonists be provided via a systemic route. Suitable routes of administration include, without limitation, oral, transdermal, inhalation, parenteral, sublingual, rectal, vaginal and intranasal. By way of example, solid oral formulations (such as tablets or capsules) providing a therapeutically effective amount of a GABA_{A} receptor agonist may be used for the inhibition of scarring. Aerosol formulations for inhalation may be preferred as means for providing GABA_{A} receptor agonists in the event that it is wished to inhibit scarring associated with healing of wounds located in the lungs and airways.

It will be appreciated that some of the routes of administration described above with reference to systemic administration may also be suitable for topical administration to a tissue in which it is wished to inhibit scarring (for example, inhalation or intranasal administration for administration of GABA_{A} receptor agonists to wounds or scars of the respiratory system).

Preferred Formulations for Use in Accordance with the Invention

Generally, medicaments of the invention may be formulated and manufactured in any form that allows for the medicament to be administered to a patient such that a therapeutically effective amount of a GABA_{A} receptor agonist, such as Gaboxadol, is provided to a site where scarring is to be prevented, reduced or inhibited.

Medicaments of the invention may preferably be provided in the form of one of more dosage units providing a therapeutically effective amount (or a known fraction or multiple of a therapeutically effective amount) of a GABA_{A} receptor agonist, such as Gaboxadol. Methods of preparing such dosage units will be well known to the skilled person; for example see Remington's Pharmaceutical Sciences 18^{th} Ed. (1990).

Compositions or medicaments containing active agents may take a number of different forms depending, in particular, on the manner in which they are to be used. Thus, for example, they may be in the form of a liquid, ointment, cream, gel, hydrogel, powder or aerosol. All of such compositions are suitable for topical application to a wound or site where a wound is to be formed, and topical application represents a preferred means of administering GABA_{A} receptor agonists to a subject (person or animal) in need of treatment.

Suitable agonists may be provided on a sterile dressing or patch, which may be used to cover a wound where scarring is to be inhibited.

GABA_{A} receptor agonists, such as Gaboxadol, may be released from a device or implant, or may be used to coat such a device e.g. a stent, or a controlled release device, or a wound dressing, or sutures for use in wound closure.

It will be appreciated that the vehicle of a composition comprising a GABA_{A} receptor agonist, such as Gaboxadol, should be one that is well tolerated by the patient and allows release of the agonist to the wound scar to be treated. Such a vehicle is preferably relatively “mild” i.e. non-inflammatory, biodegradeable, bioresolveable, or bioresorbable.

GABA_{A} receptor agonists, such as Gaboxadol, may be incorporated within a slow or delayed release device. Such devices may, for example, be placed on or inserted under the skin and the agonist may be released over days, weeks or even months.

Delayed release devices may be particularly useful for patients, such as those suffering from extensive wounding, who require long-term administration of therapeutically effective amounts of a GABA_{A} receptor agonist. Such devices may be particularly advantageous when used for the administration of an agonist that would otherwise normally require frequent administration, e.g. at least daily administration, by other routes.

A dose of a composition comprising a GABA_{A} receptor agonist, such as Gaboxadol, may preferably be sufficient to provide a therapeutically effective amount of a suitable agonist in a single administration. However, it will be appreciated that each dose need not in itself provide a therapeutically effective amount of an agonist, but that a therapeutically effective amount may instead be built up through repeated administration of suitable doses.

Various suitable forms of compositions comprising GABA_{A} receptor agonists, such as Gaboxadol, may be used in accordance with the present invention. In one embodiment a pharmaceutical vehicle for administration of a suitable agonist may be a liquid and a suitable pharmaceutical composition would be in the form of a solution. In another embodiment, the pharmaceutically acceptable vehicle is a solid and a suitable composition is in the form of a powder. In a further embodiment the GABA_{A} receptor agonist may be formulated as a part of a pharmaceutically acceptable trans-epidermal delivery system, e.g. a patch/dressing

A solid vehicle can include one or more substances that may also act as flavouring agents, lubricants, solubilizers, suspending agents, fillers, glidants, compression aids, binders or tablet-disintegrating agents; it can also comprise an encapsulating material. In powders, the vehicle is a finely divided solid that is in admixture with the finely divided agonist that will be used to inhibit scarring. In tablets, the selected agonist is mixed with a vehicle having the necessary compression properties in suitable proportions and compacted in the shape and size desired. The powders and tablets preferably contain up to 99% of a GABA_{A} receptor agonist such as Gaboxadol. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles may be used in preparing solutions, suspensions, emulsions, syrups, elixirs and pressurized compositions. The GABA_{A} receptor agonist can be dissolved or suspended in a pharmaceutically acceptable liquid vehicle such as water, an organic solvent, a mixture of both or pharmaceutically acceptable oils or fats. Phosphate buffered saline (PBS) represents an example of a preferred liquid vehicle. The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can be an oily ester such as ethyl oleate and isopropyl myristate. Sterile liquid vehicles are useful in compositions for parenteral administration. The liquid vehicle for pressurized compositions can be halogenated hydrocarbon or other pharmaceutically acceptable propellant.

Liquid pharmaceutical compositions which are sterile solutions or suspensions comprising a GABA_{A} receptor agonist, such as Gaboxadol, can be utilized by, for example, intramuscular, intrathecal, epidural, intraperitoneal, intradermal, intrastromal (cornea), intraadventitial (blood vessels) or subcutaneous injection. Sterile solutions can also be administered intravenously. The agonist may be prepared as part of a sterile solid composition that may be dissolved or suspended at the time of administration using sterile water, saline, or other appropriate sterile injectable medium (such as PBS). Vehicles are intended to include necessary and inert binders, suspending agents, lubricants and preservatives. The inventors have found that a sterile solution of a GABA_{A} receptor agonist, such as Gaboxadol, in phosphate buffered saline (PBS) represents a preferred formulation suitable for administration by injection, such as intradermal injection.

In the situation in which it is desired to administer a GABA_{A} receptor agonist by means of oral ingestion, it will be appreciated that the chosen agonist will preferably be one having an elevated degree of resistance to degradation. For example, the chosen agonist may be protected (using the techniques well known to those skilled in the art) so that its rate of degradation in the digestive tract is reduced.

Medicaments comprising a GABA_{A} receptor agonist, such as Gaboxadol, that are for use in the inhibition of scarring in the lungs or other respiratory tissues may be formulated for inhalation.

Medicaments in accordance with the invention for use in the inhibition of scarring in the body cavities e.g. abdomen or pelvis, may be formulated as an irrigation fluid, lavage, gel or instillate.

A GABA_{A} receptor agonist, such as Gaboxadol, for use in the medicaments or methods of the invention, may be incorporated in a biomaterial, from which it may be released to inhibit scarring. Biomaterials incorporating GABA_{A} receptor agonists are suitable for use in many contexts, and at many body sites, where it is desired to inhibit scarring, but may be of particular utility in providing a suitable GABA_{A} receptor agonist, such as Gaboxadol, to the eye (for example after retina surgery or glaucoma filtration surgery), or to sites where it is wished to inhibit restenosis or adhesions. The inventors believe that biomaterials incorporating GABA_{A} receptor agonists may be used in the manufacture of sutures, and such sutures represent a preferred embodiment of a medicament of the invention.

Known procedures, such as those conventionally employed by the pharmaceutical industry (e.g. in vivo experimentation, clinical trials etc), may be used to establish specific formulations of compositions comprising GABA_{A} receptor agonists, such as Gaboxadol, and precise therapeutic regimes for administration of such compositions (such as the required daily doses of the agonist, and the preferred frequency of administration).

Medicaments or methods of the invention may be used to inhibit scarring as a monotherapy (e.g. through use of medicaments or methods of the invention alone). Alternatively the methods or medicaments of the invention may be used in combination with other compounds or treatments for the inhibition of scarring. Suitable compounds that may be used as parts of such combination therapies will be well known to those skilled in the art.

It will be appreciated that many of the advantages that may be gained as a result of inhibiting scarring of humans are also applicable to other animals, particularly veterinary or domestic animals (e.g. horses, cattle, dogs, cats etc). Accordingly it will be recognised that the medicaments and methods of the invention may also be used inhibit scarring of non-human animals.

The invention will now be further described with reference to the accompanying Sequence Information, Experimental Results, and Figures, in which:

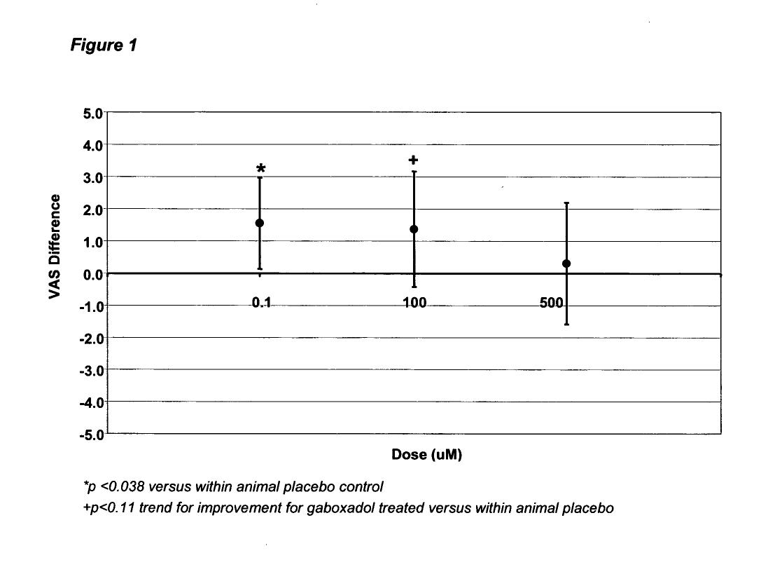

FIG. 1 is a graph comparing the mean differences between macroscopic VAS scores of scars formed on healing of wounds treated with either the GABA_{A} receptor agonist Gaboxadol hydrochloride or with diluent control (PBS). Scores were produced 70 days after wounding. Filled circles show the difference values (calculated as VAS_{diluent control}−VAS_{treated}) with bars illustrating the ±95% confidence interval (n=6 per treatment group). “*” indicates a statistically significant inhibition of scarring based on the difference in VAS scores (p<0.038), while “+” indicates a trend towards inhibition of scarring (p=0.11). The values shown correspond to scars from wounds treated with two administrations of 100 μl of (from left to right) a 0.1 μM solution of Gaboxadol hydrochloride, a 100 μM solution of Gaboxadol hydrochloride, and a 500 μM solution of Gaboxadol hydrochloride

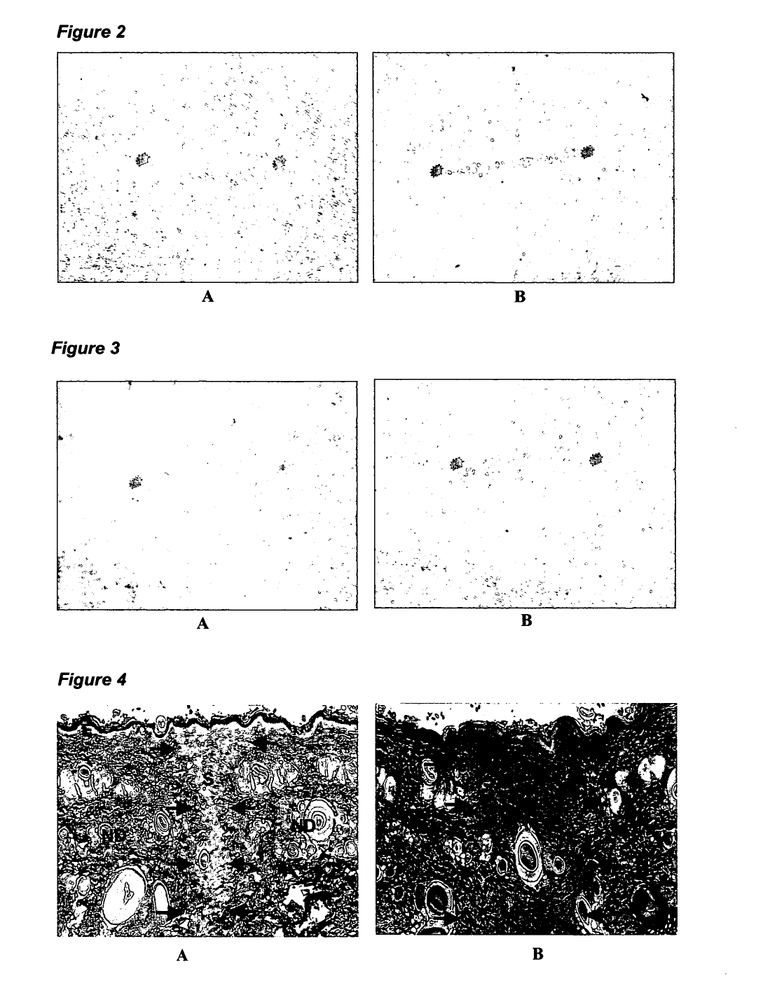

FIG. 2 compares representative images showing the macroscopic appearance of a treated scar (produced on healing of a wound treated with a total of 3.52 μg of the GABA_{A} receptor agonist Gaboxadol hydrochloride, by means of two administrations of 100 μl of a 0.1 μM solution of Gaboxadol hydrochloride), on the left, with a scar produced on the healing of a diluent control wound on the right.

FIG. 3 compares representative images showing the macroscopic appearance of a treated scar (produced on healing of a wound treated with a total of 3.52 μg of the GABA_{A} receptor agonist Gaboxadol hydrochloride, by means of two administrations of 100 μl of a 0.1 μM solution of Gaboxadol hydrochloride), on the left, with a scar produced on the healing of a diluent control wound on the right.

FIG. 4 compares representative images showing the microscopic appearance of a treated scar (produced on healing of a wound treated with a total of 3.52 ng of the GABA_{A} receptor agonist Gaboxadol hydrochloride, by means of two administrations of 100 μl of a 0.1 μM solution of Gaboxadol hydrochloride), on the left, with a scar produced on the healing of a diluent control wound on the right. The images were taken, at ×5 magnification, from histological sections of scars stained with Masson's Trichrome. Arrows indicate the edges of resultant scars following full thickness incisional wounding and treatment. ‘E’ indicates epidermis, ‘S’ indicates the scar, ‘ND’ indicates the normal dermis surrounding the scar.

### EXPERIMENTAL RESULTS

The GABA_{A} receptor agonist Gaboxadol hydrochloride (Sigma Aldrich, catalogue number T101) was diluted in phosphate buffered saline (PBS) to produce medicaments of the invention having the following concentrations:

i) 1.76 ng/100 μL (a concentration of 0.1 μM, in which each 10 μl of the medicament provides 10 pmoles of Gaboxadol hydrochloride);
ii) 1.76 μg/100 μL (a concentration of 100 μM, in which each 100 μl of the medicament provides 10 nmoles of Gaboxadol hydrochloride); and
iii) 8.83 μg/100 μL (a concentration of 500 μM, in which each 100 μl of the medicament provides 50 nmoles of Gaboxadol hydrochloride).

### Scarring Model

At day 0, male Sprague Dawley rats (200-250 g) were anaesthetised, shaved and wound sites were marked according to a template in which two wounds, each 1 cm in length, were made at sites 5 cm from the base of the skull and 1 cm from the midline on the back of each rat.

One hundred microlitres of either one of the medicaments of the invention (comprising either 0.1 μM, 100 μM or 500 μM Gaboxadol hydrochloride in phosphate buffered saline pH 7.2—purchased from GIBCO BRL, Cat. # 20012-019), or placebo (PBS only) was injected intradermally at the wound sites. The intradermal injections caused the formation of a raised bleb, which was then immediately incised to form 1 cm long experimental wounds.

All treated or diluent control wounds were re-injected again 1 day post-wounding with the appropriate treatment (drug or placebo) via injection of 50 μl to each of the two margins of the 1 cm wound. All wounds were all harvested at day 70 post-wounding.

### Assessment of Scarring

The wounds were photographed after wounding, prior to re-injection on day 1 and on day of harvest. The scars were assessed using a visual analogue scale (VAS) consisting of a 0-10 cm line representing a scale, from left to right, of 0 (corresponding to normal skin) to 10 (indicative of a bad scar).

For microscopic assessment the scars were excised from the experimental rats (incorporating a small amount of surrounding normal tissue) and fixed in 10% (v/v) buffered formal saline. The fixed tissue was then processed for wax histology, stained using Masson's trichrome, and scarring in the regenerated dermis was assessed.

### Results

Treatments comprising two sequential intradermal injections of 100 μl of a solution of Gaboxadol hydrochloride in PBS at a concentration of 0.1 μM, 100 μM or 500 μM (respectively providing 1.76 ng, 1.76 μg and 8.83 μg of Gaboxadol per centimetre in each administration) improved the macroscopic appearance of scars resulting from full thickness cutaneous incisional wounds, in comparison to scars resulting from diluent control wounds. The improvement obtained was greatest when the GABA_{A} receptor agonist Gaboxadol was administered at 1.76 ng or 1.76 μg per centimetre (in this case via administration of the 0.1 μM or 100 μM solutions), and the beneficial results achieved can be seen in the representative images shown in FIGS. 2 and 3 (results obtained using solutions of 0.1 μM or 100 μM Gaboxadol hydrochloride respectively).

The treatment using 0.1 μM Gaboxadol hydrochloride resulted in a statistically significant inhibition of scarring as assessed with reference to the macroscopic appearance of treated wounds (p<0.05 as compared to diluent controls). Treatment with 100 μM Gaboxadol hydrochloride resulted in a trend towards scar reduction (p=0.11 as compared to diluent controls). These results can be seen in accompanying FIG. 1.

Treatment of wounds with the GABA_{A} receptor agonist Gaboxadol hydrochloride in a solution of 0.1 μM, 100 μM or 500 μM in PBS also lead to a reduction of scarring when assessed microscopically, as can be seen in the representative results obtained with a 0.1 μM solution and shown in FIG. 4.

These results clearly illustrate the ability of GABA_{A} receptor agonists, such as Gaboxadol hydrochloride, to achieve in vivo prevention, reduction or inhibition of scarring formed on healing of a wound. The results provide an indication of therapeutically effective amounts of such GABA_{A} receptor agonists that may be provided to a wound, or site where a wound is to be formed, to inhibit scarring.

1-22. (canceled)

24. A medicament, comprising:
a. an agonist of a GABA_{A} receptor, wherein said GABA_{A} receptor agonist is useful for the prevention, reduction or inhibition of scarring formed on healing of a wound.

25. The medicament according to claim 24, wherein said GABA_{A} receptor agonist is an agonist specific for the GABA_{A} receptor.

26. The medicament according to claim 24, wherein said GABA_{A} receptor agonist comprises 7-tetra hydroisoxazolo[5,4-c]pyridin-3-ol, or a pharmaceutically acceptable salt thereof.

27. The medicament according to claim 24, wherein said GABA_{A} receptor agonist is suitable for application at a site where scarring is to be prevented, reduced or inhibited.

28. The medicament according to claim 24, wherein said GABA_{A} receptor agonist is suitable for application to an area of the body selected from the group consisting of: a site where a wound is to be formed; and a wound.

29. The medicament according to claim 24, wherein the scarring includes scarring formed upon healing of a wound of the dermis.

30. The medicament according to claim 24, wherein the wound is a surgical wound.

31. The medicament according to claim 24, wherein the surgical wound is associated with scar revision surgery.

32. The medicament according to claim 24, wherein the scarring comprises scarring formed on healing of a wound selected from the group consisting of: wounds of the eye, wounds resulting from eye surgery, wounds resulting from LASIK surgery, wounds resulting from LASEK surgery, wounds resulting from PRK surgery, wounds resulting from glaucoma filtration surgery, wounds resulting from cataract surgery; wounds subject to capsular contraction; wounds resulting from retinal surgery; wounds of blood vessels; wounds of tendons, ligaments or muscle; wounds of the oral cavity, wounds of the lips; wounds of the palate; and wounds of body cavities such as the abdominal cavity, pelvic cavity and thoracic cavity.

33. The medicament according to claim 24, wherein said GABA_{A} receptor agonist useful for the prevention, reduction or inhibition of scarring formed on the healing of a wound when said GABA_{A} receptor agonist is administered an amount of between about 20 fmoles and about 100 nmoles, per centimetre of a site to which it is provided.

34. The medicament according to claim 24, wherein the use of said GABA_{A} receptor agonist comprises the localised administration of said GABA_{A} receptor agonist.

35. The medicament according to claim 24, wherein the use said GABA_{A} receptor agonist comprises the administration of said GABA_{A} receptor agonist by means of an intradermal injection.

36. The medicament according to claim 3, wherein said 7-tetra hydroisoxazolo[5,4-c]pyridin-3-ol, or a pharmaceutically acceptable salt thereof is useful for the prevention, reduction or inhibition of scarring formed on the healing of a wound when said 7-tetra hydroisoxazolo[5,4-c]pyridin-3-ol, or a pharmaceutically acceptable salt thereof is administered an amount of between about 20 fmoles and about 100 nmoles, per centimetre of a site to which it is provided.

37. A method of preventing, reducing or inhibiting scarring formed on healing of a wound, comprising the steps of:
a. administering a therapeutically effective amount of an agonist of a GABA_{A} receptor to a patient in need of such prevention, reduction or inhibition.

38. The method according to claim 37, wherein said GABA_{A} receptor agonist is an agonist specific to the GABA_{A} receptor.

39. The method according to claim 37, wherein said GABA_{A} receptor agonist comprises 7-tetra hydroisoxazolo[5,4-c]pyridin-3-ol, or a pharmaceutically acceptable salt thereof.

40. The method according to any of claim 37, wherein said GABA_{A} receptor agonist is applied at an area of the body wherein scarring is to be prevented, reduced or inhibited.

41. The method according to claim 37, wherein the site to which said GABAA receptor agonist is applied to an area of the body selected from the group consisting of: a site where a wound is to be formed; and a wound.

42. The method according to claim 37, wherein the scarring comprises scarring formed on healing of a wound of the dermis.

43. The method according to claim 37, wherein the scarring comprises scarring formed on healing of a wound selected from the group consisting of: wounds of the eye, wounds resulting from eye surgery, wounds resulting from LASIK surgery, wounds resulting from LASEK surgery, wounds resulting from PRK surgery, wounds resulting from glaucoma filtration surgery, wounds resulting from cataract surgery; wounds subject to capsular contraction; wounds of blood vessels; wounds of tendons, ligaments or muscle; wounds of the oral cavity, wounds of the lips; wounds of the palate; and wounds of body cavities such as the abdominal cavity, pelvic cavity and thoracic cavity.

44. The method according to claim 37, wherein the wound is a surgical wound.

45. The method according to claim 37, wherein the GABA_{A} receptor agonist is administered in an amount of between about 10 pmoles and 200 nmoles, per centimetre of a site to which it is administered.

46. The method according to claim 39, wherein said 7-tetra hydroisoxazolo[5,4-c]pyridin-3-ol, or a pharmaceutically acceptable salt is administered in an amount of between about 10 pmoles and 200 nmoles, per centimetre of a site to which it is administered.
